(19) United States
(12) Patent Application Publication
(10) Pub. No.: US 2008/0221467 A1
(43) Pub. Date: Sep. 11, 2008

(54) Method and systms for regional assessment of pulmonary function

(75) Inventors: Surik Papyan, Haifa (IL); Konstantin Goulitski, Holon (IL)

Correspondence Address: NATH & ASSOCIATES, 112 South West Street, Alexandria, VA, 22314, US

(73) Assignee: DeepBreeze Ltd., Industrial Park Or Akiva (IL)

(21) Appl. No.: 12/068,344

(22) Filed: Feb. 5, 2008

Related U.S. Application Data
(60) Provisional application No. 60/899,666, filed on Feb. 6, 2007.

(51) Int. Cl. A61B 7/00; A61B 5/08

(52) U.S. Cl. 600/529; 600/586

(57) ABSTRACT

A method and system for regional assessment in two or more regions of an individual's lungs. The system includes a plurality of transducers configured to be fixed over the thorax. Each transducer generates a signal P(xᵢ,t) indicative of pressure waves at the location of the transducer. The transducers are divided into subsets, where each subset overlies a specific region of the two or more regions. An energy assessment signal is calculated from each of the signals P(xᵢ,t). For each region, an assessment of the region is calculated from the energy assessment signals of the region.

This application claims the benefit of prior U.S. provisional patent application No. 60/899,666 filed Feb. 6, 2007, the contents of which are hereby incorporated by reference in their entirety.

### FIELD OF THE INVENTION

This invention relates to medical devices and methods, and more particularly to such devices and methods for analyzing body sounds.

### BACKGROUND OF THE INVENTION

Regional assessment of lung physiology has been carried out using radionucleotide perfusion also known as the “VQ scan”. In this technique, radioactive particles are either injected into the subject's blood system or the subject is allowed to inhale suspended radioactive particles. X-ray images of the lungs are obtained and one or both of the lungs in the image is divided into two or more regions. A separate analysis of each lung region is then performed. In most regional lung assessments, each of the two lung images is divided into three parts (top, middle and bottom), and an assessment of lung function or physiology in each region is obtained. Typically, regional assessment involves determining the fraction of the total detected radioactivity detected in each region. The amount of radioactivity detected in each part may be correlated with the lung condition in each part.

Body sounds are routinely used by physicians in the diagnosis of various disorders. A physician may place a stethoscope on a person's chest or back and monitor the patient's breathing in order to detect adventitious (i.e. abnormal or unexpected) lung sounds. The identification and classification of adventitious lung sounds often provides important information about pulmonary abnormalities.

It is also known to fix one or more microphones onto a subject's chest or back and to record lung sounds. U.S. Pat. No. 6,139,505 discloses a system in which a plurality of microphones are placed around a patient's chest. The recordings of the microphones during inhalation and expiration are displayed on a screen, or printed on paper. The recordings are then visually examined by a physician in order to detect a pulmonary disorder in the patent. Kompis et al. (Chest, 120(4), 2001) disclose a system in which M microphones are placed on a patient's chest, and lung sounds are recorded. The recordings generate M linear equations that are solved using a least-squares fit. The solution of the system is used to determine the location in the lungs of the source of a sound detected in the recordings.

U.S. Pat. No. 6,887,208 to Kushnir et al., provides a system and method for recording and analyzing sounds produced by the respiratory tract. Respiratory tract sounds are recorded at a plurality of locations over an individual's thorax and the recorded sounds are processed to produce an image of the respiratory tract. The processing involves determining from the recorded signals an average acoustic energy, at a plurality of locations over the thorax over a time interval from t₁ to t₂. The term “acoustic energy” at a location is used herein to refer to a parameter indicative of or approximating the product of the pressure and the mass propagation velocity at that location. The image may be used to analyze respiratory tract physiology and to detect pathological conditions. Additionally, a time interval can be divided into a plurality of sub-intervals, and an average acoustic energy determined over the thorax for two or more of the sub-intervals. An image of each of these sub intervals may then be determined and displayed sequentially on a display monitor. This generates a movie showing dynamic changes occurring in the acoustic energy in the respiratory tract over the time interval.

### SUMMARY OF THE INVENTION

The present invention provides a system and method for regional assessment of lung functioning. In accordance with the invention, microphones are affixed to the body surface at a plurality of locations over the thorax, and signals indicative of lung sounds at the location of each transducer are recorded. Each signal is analyzed in order to produce an energy assessment signal at the location of each transducer. The set of transducers is clustered into subsets, where each subset consists of transducers located on the body surface overlying a particular region of the lungs. The regions may correspond to anatomical regions of the lungs, or may be determined independently of the lung anatomy. For each subset of locations, a regional assessment of the underlying lung region is obtained based upon the energy assessment signals. The regional assessment may be dynamic, i.e. an assessment that varies in at least a portion of the breathing cycle. In this case the regional assessment may be, for example, signals calculated as the sum of the assessment signals at the location in each subset, the maximum signal, the minimum signal or an average signal. The regional assessment may be the sum of the values of an energy assessment (dynamic or static) at the locations in the subset divided by the sum of the values of the energy assessment signals of the entire set of transducers. The regional assessment may be a non-dynamic or overall assessment. In this case, the regional assessment may be an average value of a dynamic regional assessment over at least a portion of a breathing cycle. In one embodiment, each lung is divided into three regions (top, middle and bottom), and a regional assessment is obtained as explained above for each of the six regions. In another embodiment, the lungs are divided into regions so that each region has the same number of overlying microphones. The regional assessment may be presented in the form of a table. Alternatively, a diagram showing the contours of the lungs and the lung regions is generated, with the value of the regional assessment of each region appearing in that region of the diagram.

In one embodiment of the invention, a breathing cycle is divided into two or more time intervals, and a regional assessment of the lungs, is obtained in accordance with the invention for each time interval.

The system of the invention includes a plurality of N transducers (microphones) configured to be attached to an essentially planar region R of the individual's back or chest over the individual's thorax. The transducers are typically embedded in a matrix that permits to affix them easily on the individual's skin. Such a matrix may typically be in the form of a vest or garment for easily placing over the individual's thorax. As may be appreciated, different matrices may be used for differently sized individuals; for different ages, sexes, etc.

In one embodiment, the parameter calculated at each of the plurality locations is an average acoustical energy. The term “acoustic energy” at a location is used herein to refer to a parameter indicative of or approximating the product of the pressure and the mass propagation velocity at that location. In a most preferred embodiment of the invention, an image of the lungs is generated from the calculated average acoustic energies. The image is displayed on a display device with the lungs in the image being divided into the lung regions. The regional assessment of the lung regions is displayed together with the image of the lungs.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

Thus, in one of its aspects, the invention provides a system for regional assessment in two or more regions of an individual's lungs comprising:
(a) a plurality of N transducers, where n is an integer greater than or equal to 2, each transducer configured to be fixed on a surface of the individual over the thorax, the ith transducer being fixed at a location xᵢ and generating a signal P(xᵢ,t) indicative of pressure waves at the location xᵢ; for i=1 to N; the transducers being divided into subsets, each subset overlying a specific region of the two or more regions; and
(b) a processor configured to:
   (i) receive the signals P(xᵢ,t) obtained over a time period and calculate from each signal P(xᵢ,t) an energy assessment signal at the location xᵢ; and
   (ii) to calculate, for each of the two or more regions, an assessment of the lungs in a calculation involving the energy assessment signals obtained by transducers overlying the region.

In another of its aspects, the invention provides a method for regional assessment in two or more regions of an individual's lungs comprising:
(a) receiving a plurality of N signals P(xᵢ,t), where N is an integer greater than or equal to 2, each signal being generated by a transducer fixed on a surface of the individual over the thorax, the ith transducer being fixed at a location xᵢ and generating a signal P(xᵢ,t) indicative of pressure waves at the location xᵢ; for i=1 to N; the transducers being divided into subsets, each subset overlying a specific region of the two or more regions, and the signals P(xᵢ,t) being obtained over a time period;
(b) calculating from each signal P(xᵢ,t) an energy assessment signal at the location xᵢ; and
(c) calculating, for each of the two or more regions, an assessment of the lungs in a calculation involving the energy assessment signals obtained by transducers overlying the region.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for performing a regional assessment of lung function in accordance with one embodiment of the invention;

FIG. 2 shows a method for performing a regional assessment of lung function in accordance with one embodiment of the invention;

FIG. 3 shows a method for calculating an energy assessment signal for use in the method of FIG. 2;

FIG. 4 shows a method for calculating an energy assessment signal;

FIG. 5 shows a method for calculating a regional assessment of respiratory function using energy assessment signals;

FIG. 6 shows placement of sound transducers over a subject's lungs;

FIG. 7 shows the functions σ_{fs}^{norm} (xᵢ,k) for transducers overlying the left lung (FIG. 7a) and the right lung (FIG. 7b) of an individual;

FIG. 8 shows the functions R_{σ}(xᵢ,k) for the transducers overlying the left lung (FIG. 8a) and the transducers overlying the right lung (FIG. 8b);

FIG. 9 shows the function R̅_̅{̅σ̅}̅^̅{̅L̅}̅(̅k̅)̅;

FIG. 10 shows the functions K^{L}(k) (curve (a)) and K^{Ld}(k) (curve (b));

FIG. 11 shows regional assessment of the left lung divided into three regions, the regional assessment of the top region (curve (a)), the regional assessment of the middle region (curve (b)) and the regional assessment of the bottom region (curve (c)); and

FIG. 12 shows regional assessment of the right lung divided into three regions, the regional assessment of the top region (curve (a)), the regional assessment of the middle region (curve (b)) and the regional assessment of the bottom region (curve (c));

### DETAILED DESCRIPTION OF THE INVENTION

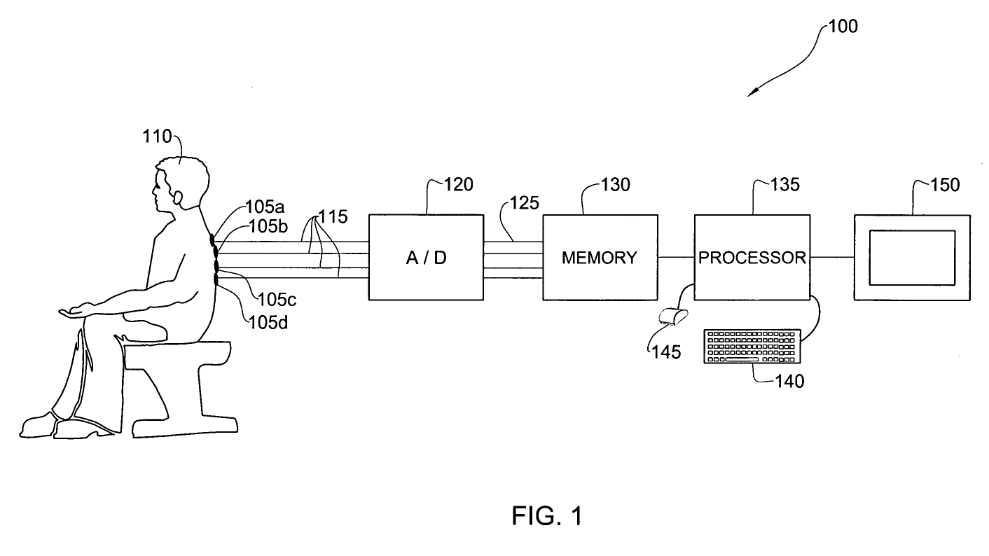

FIG. 1 shows schematically a system generally indicated by 100 for performing regional assessment of the lungs in accordance with one embodiment of the invention. The system 100 includes a plurality of N sound transducers, where N is an integer greater than or equal to 2. Four transducers 105a, 105b, 105c, and 105d are shown in FIG. 1. This is by way of example only, and the system and method of the invention may be carried out using a transducer array having any number of transducers greater than or equal to two. The transducers 105 may be any type of sound transducer, such as a microphone or Doppler shift detector.

The set of the transducers 105 are configured to be attached to an essentially planar region R of the back or chest of an individual 110 overlying the individual's lungs. The transducers 105 may be applied to the subject by any means known in the art, for example, using an adhesive, suction, or fastening straps. The transducers may be embedded in a matrix that permits them to be affixed easily onto the individual's skin. Such a matrix may be in the form of a vest or garment for easily placing over the individual's thorax. As may be appreciated, different matrices may be used for differently sized individuals, for different ages, sexes, etc.

Positions in the region R are indicated by two-dimensional position signals x=(x₁,x₂) in a two-dimensional coordinate system defined in the planar region R. The ith transducer, for i=1 to N, is fixed at a position xᵢ in the region R and generates a respective analog voltage signal 115, denoted herein by P(xᵢ,t) indicative of pressure waves in the body arriving at the location xᵢ.

The transducers 105 are divided into at least two subsets, where each subset consists of transducers overlying a specific region of the lungs. For example, the transducers may be divided into two subsets, where one subset overlies the left lung and the other subset overlies the right lung. As another example, each lung may be divided into 3 regions (top, middle, and bottom) and the transducers divided into six subsets (left lung top region, left lung middle region, left lung bottom region, right lung top region, right lung middle region, and right lung bottom region).

The analog signals 115 are digitized by a multichannel analog to digital converter 120 to generate respective digital data signals S(xᵢ,t) 125. The data signals 125 are input to a memory 130. Data input to the memory 130 are accessed by a processor 135 configured to process the data signals 125.

An input device, such as a computer keyboard 140 or mouse 145, is used to input relevant information relating to the examination such as personal details of the individual 110. The input device 140 may also be used to input values of one or more times t₁ and t₂ that specify times at which the signals S(xᵢ,t) are to be analyzed or that specify one or more time intervals over which no signals S(xᵢ,t) are to be analyzed. The system 100 may further comprise a display device 150 for displaying the results of the regional assessment.

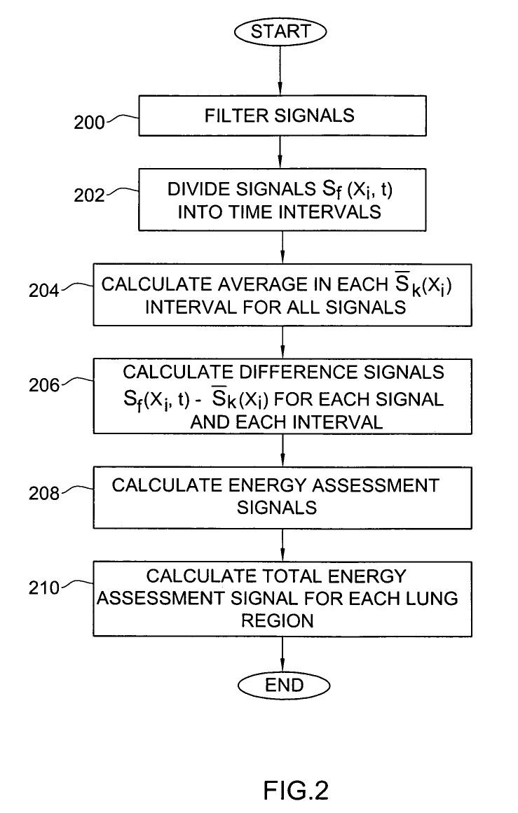

FIG. 2 shows a method for performing regional assessment of lung function from the signals S(xᵢ,t) 125 carried out by the processor 135 in accordance with one embodiment of the invention. In step 200, the signals S(xᵢ,t) 125 are filtered to produce respective filtered signals S_{f}(xᵢ,t) in order to remove one or more components of the signals which do not arise from respiratory tract sounds, such as cardiovascular sounds. Respiratory tract sounds are typically in the range of 100 to 2000 Hz while cardiac sounds are in the range of 8 to 70 Hz. Thus, cardiac sounds can be removed from the signal by band pass filtering in the rage of 180-350 Hz. This band pass filtering also removes from the signals artifacts and adventitious lung sounds.

In step 202, each of the signals S_{f}(xᵢ,t) is divided into time intervals by a time window, and in step 204 the average value S̅ₖ(xᵢ) of each signal in each interval is calculated, where

$${\overline{S}}_{k} ( {x}_{i} ) = \frac{1}{n} {\sum }_{j = 1}^{n} {S}_{f} ( {x}_{i} , {t}_{j} )$$

where k is the interval number, tⱼ are the time samples in the interval and n is the number of samples in the interval. In step 206, a difference signal S_{f}(xᵢ,t)− S̅ₖ(xᵢ) is calculated. In step 208, an energy assessment signal is calculated in a calculation involving the difference signals S_{f}(xᵢ,t)− S̅ₖ(xᵢ). The calculation of the energy assessment signal may involve the algebraic expression |S_{f}(xᵢ,t)− S̅ₖ(xᵢ)| or the expression |S_{f}(xᵢ,t)− S̅ₖ(xᵢ)|^{p} where p is a predetermined constant. In a presently preferred embodiment, p=2. In an even most preferred embodiment, as described below, the energy signal is calculated using the standard deviation of the signal S_{f}(xᵢ,t) in each interval k. In step 210, for each lung region, the sum of the energy assessment signals of the transducer subset overlying the region is calculated.

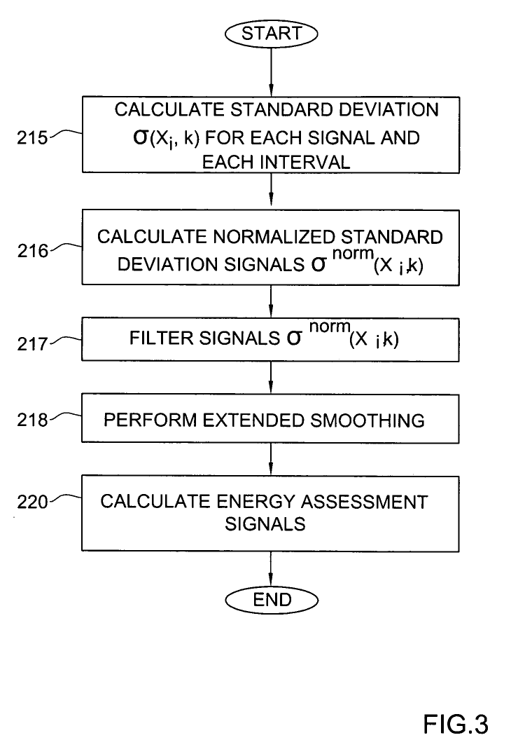

As explained above, in a most preferred embodiment, the energy assessment signals are calculated using the standard deviations of the signals S_{f}(xᵢ,t). FIG. 3 shows a method for calculating an energy assessment signal in step 208 of FIG. 2, in accordance with this embodiment. In step 215, the standard deviation σ(xᵢ,k) for each interval is calculated, where

$$σ ( {x}_{i} , k ) = {( \frac{1}{n} {\sum }_{j = 1}^{n} {( {S}_{f} ( {x}_{i} , {t}_{j} ) - {\overline{S}}_{k} ( {x}_{i} ) )}^{2} )}^{\frac{1}{2}} \qquad ( 2 )$$

where, as above, k is the interval number, tⱼ is a time sample in the interval, n is the number of samples in the interval, and S̅ₖ(xᵢ) is the average value of the signal in the interval.

A normalized standard deviation signal σ^{norm}(xᵢ,k) is then calculated in step 216, where

σ^{norm}(xᵢ,k)=σ(xᵢ,k)/ σ̅, k=1 . . . nₖ

where σ̅ is the average value of the standard deviation calculated for all of the intervals:

$$\overline{σ} = \frac{1}{{n}_{k}} {\sum }_{k = 1}^{{n}_{k}} σ ( {x}_{i} , k ) \qquad ( 4 )$$

where nₖ is the number of intervals.

Next, in step 217, the signals σ^{norm}(xᵢ,k) are preferably filtered. The filtering is preferably a one-dimensional median filtering, for example, as performed by using the MATLAB algorithm “medfilt”. This generates filtered normalized standard deviation signals σ_{f}^{norm}(xᵢ,k), for k=1 to nₖ. In step 218 extended smoothing is preferably performed on the signals σ_{f}^{norm}(xᵢ,k), for example, using the MATLAB algorithm “filtfilt. This produces filtered and smoothed normalized sequences σ_{fs}^{norm}(xᵢ,k). This tends to remove impulse artifacts (“clicks”) introduced into the signal by ambient noise.

In step 220, an energy assessment signal is calculated for each transducer location xᵢ and for each time interval in a calculation involving the respective filtered and smoothed normalized sequence σ_{fs}^{norm}(xᵢ,k).

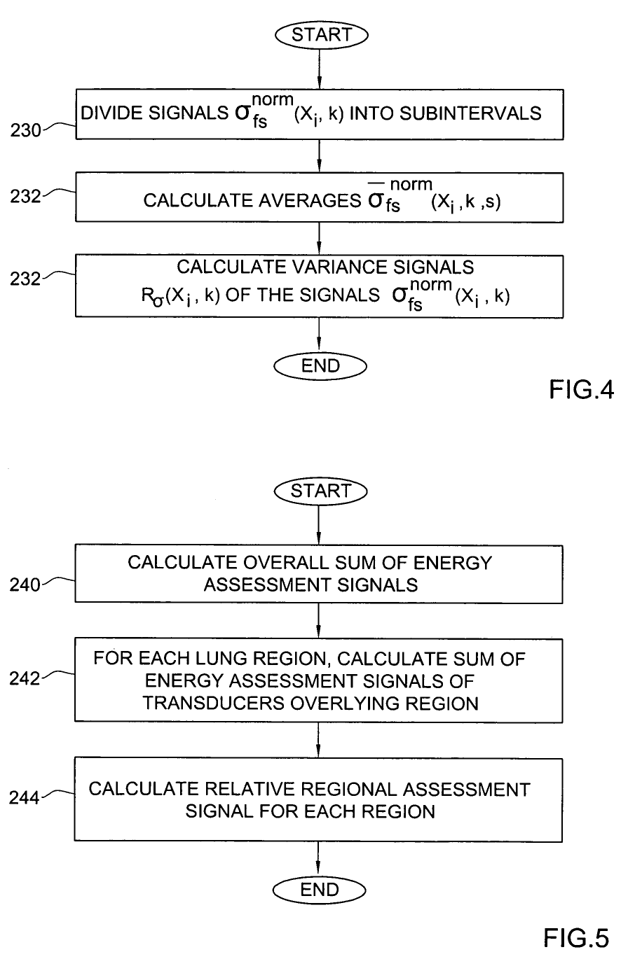

Any method for calculating the energy assessment signal from the respective filtered and denoised signal σ_{fs}^{norm}(xᵢ,k) may be used in step 220 of the algorithm of FIG. 3. FIG. 4 shows a presently preferred method for calculating an energy assessment signal from a filtered and denoised signal σ_{fs}^{norm}(xᵢ,k). In step 230, the signal σ_{fs}^{norm}(xᵢ,k) is divided into one or more subintervals by a sliding window having nₛ samples. In step 232, the average value of each signal σ_{fs}^{norm}(xᵢ,k) in each subinterval σ̅_{fs}^{norm}(xᵢ,k,s) is calculated, where σ̅_{fs}^{norm}(xᵢ,k,s) is the average value of the signal σ_{fs}^{norm}(xᵢ,k) in the subinterval s of the interval k,

$${\overline{σ}}_{fs}^{norm} ( {x}_{i} , k , s ) = \frac{1}{{n}_{s}} {\sum }_{j = 1}^{{n}_{s}} {σ}_{fs}^{norm} ( {x}_{i} , k , s , {t}_{j} ) ,$$

wherein the tⱼ are the values of the signal σ_{fs}^{norm}(xᵢ,k) in the subinterval s. Finally, in step 234, an energy assessment signal is calculated for each subinterval s in the interval k of each signal σ_{fs}^{norm}(xᵢ,k). In this embodiment, the energy assessment signals R_{σ}(xᵢ,k) are calculated for each transducer as the variance of σ_{fs}^{norm}(xᵢ,k):

$${R}_{σ} ( {x}_{i} , k ) = {\sum }_{s = 1}^{{n}_{s}} {( {σ}_{fs}^{norm} ( {x}_{i} , k , s ) - {\overline{σ}}_{fs}^{norm} ( {x}_{i} , k , s ) )}^{2} \qquad ( 6 )$$

The regional assessment may be performed by calculating for each transducer subset the sum of the energy assessment signal for each transducer in the subset over at least a portion of the breathing cycle. This produces a dynamic regional assessment that varies over the at least portion of the breathing cycle.

FIG. 5 shows a method for calculating a dynamic regional assessment of respiratory function using the energy assessment signals R_{σ}(xᵢ,k) in accordance with the invention. In step 240, for each interval k, the overall sum of the energy assessment signals for all of the transducers is calculated as:

$${R}_{σ} ( k ) = {\sum }_{i = 1}^{N} {R}_{σ} ( {x}_{i} , k ) \qquad ( 9 )$$

Then, in step 242, for each region of the lungs, a regional assessment signal is obtained by calculating the sum of the energy assessment signals of each transducer in the subset of the transducers overlying the region. In step 244, the relative regional assessment signal for each region is obtained by dividing the regional assessment signal of the region obtained in step 242 by the total signal is by R_{σ}(k) calculated in step 240.

For example, when the set of transducers is divided into two subsets, one subset overlying the left lung (the “L” subset) and the other subset overlying the right lung (the “R” subset), sum of the left and right lung energy assessment signals are obtained as follows:

$$\begin{matrix}{R}_{σ}^{L} ( k ) = {\sum }_{x i ∈ L} {R}_{σ} ( {x}_{i} , k ) & ( 10 ) \\ {R}_{σ}^{R} ( k ) = {\sum }_{X i ∈ R} {R}_{σ} ( {x}_{i} , k ) & ( 11 )\end{matrix}$$

where L and R are the set of transducers overlying the left and right lung, respectively.

The left and the right lung regional assessment signals R̅_̅{̅σ̅}̅^̅{̅L̅}̅(̅k̅)̅ and R̅_̅{̅σ̅}̅^̅{̅R̅}̅(̅k̅)̅, respectively, are then obtained by dividing R_{σ}^{L}(k) and R_{σ}^{R}(k), respectively by R_{σ}(k):

$$\overline{{R}_{σ}^{L} ( k )} = \frac{{R}_{σ}^{L} ( k )}{{R}_{σ} ( k )} , \overline{{R}_{σ}^{R} ( k )} = \frac{{R}_{σ}^{R} ( k )}{{R}_{σ} ( k )} \qquad ( 12 )$$

This produces a dynamic regional assessment, which varies over time during the breathing cycle.

An overall regional assessment may be calculated from the dynamic regional assessment. In a preferred embodiment, the average of the gradients of R̅_{σ}^{L}(k,s) and R̅_{σ}^{R}(k,s) are calculated:

$$grad {R}_{σ}^{L} ( k ) = \frac{1}{{n}_{d} - 1} {\sum }_{j = 1}^{{n}_{d} - 1} \vert {\overline{R}}_{σ}^{L} ( k , {t}_{j + 1} ) - {\overline{R}}_{σ}^{L} ( k , {t}_{j} ) \vert$$

$$grad {R}_{σ}^{R} ( k ) = \frac{1}{{n}_{d} - 1} {\sum }_{j = 1}^{{n}_{d} - 1} \vert {\overline{R}}_{σ}^{R} ( k , {t}_{j + 1} ) - {\overline{R}}_{σ}^{R} ( k , {t}_{j} ) \vert$$

Vectors K^{L}(k), K^{R}(k), referred to herein as the left and right “criterion vector”, respectively, is then calculated where

K^{L}(k)=grad R_{σ}^{L}(k)·σ^{L}(k)σ^{R}(k) and

K^{R}(k)=grad R_{σ}^{R}(k)·σ^{L}(k)σ^{R}(k) where

$${σ}^{L} ( k ) = {\sum }_{X i ∈ L} \frac{\vert {σ}_{fs}^{norm} ( {x}_{i} , k ) - {\overline{σ}}_{fs}^{norm} ( {x}_{i} , k ) \vert}{\vert {σ}_{fs}^{norm} ( {x}_{i} , k ) - {\overline{σ}}_{fs}^{norm} ( {x}_{i} , k ) \vert}$$

$${σ}^{R} ( k ) = {{\sum }^{{N}_{L}}}_{X i ∈ R} \frac{\vert {σ}_{fs}^{norm} ( {x}_{i} , k ) - {\overline{σ}}_{fs}^{norm} ( {x}_{i} , k ) \vert}{\vert {σ}_{fs}^{norm} ( {x}_{i} , k ) - {\overline{σ}}_{fs}^{norm} ( {x}_{i} , k ) \vert}$$

where, as before, L and R are the sets of transducers overlying the left and right lung, respectively.

The vectors K^{L}(k,s) and K^{R}(k,s) are now divided into intervals by a sliding window of length n_{d} and sliding step equal to 1. The average of the gradient of the vectors K^{L}(k,s) and K^{R}(k,s) are calculated

$${K}^{Ld} ( k ) = \frac{1}{{n}_{d} - 1} {\sum }_{j = 1}^{{n}_{d} - 1} \vert {K}^{L} ( k , {t}_{j + 1} ) - {K}^{L} ( k , {t}_{j} ) \vert$$

$${K}^{Rd} ( k ) = \frac{1}{{n}_{d} - 1} {\sum }_{j = 1}^{{n}_{d} - 1} \vert {K}^{R} ( k , {t}_{j + 1} ) - {K}^{R} ( k , {t}_{j} ) \vert$$

For the regional assessment of the left lung, subregions S are found in which the value of K^{Ld}(k) is below the median value of the vector K^{Ld}(k). The longest contiguous region of the interval k below the median value is preferably extended by a few flanking samples at each end. The region is a stable region and characterizes the respiration. The average of R_{σ}^{L}, R̅_̅{̅σ̅}̅^̅{̅L̅}̅ is calculated on this interval.

If the average R̅_̅{̅σ̅}̅^̅{̅L̅}̅ this average is above a predetermined number, for example, 55, then R^{Lx} is defined as the maximum of R_{σ}^{L} in this interval. Otherwise R^{Lx} is defined as the minimum of R_{σ}^{L} in this interval. The overall regional assessment P^{L} for the left lung is then defined as

$${P}^{L} = \frac{{R}^{{L}^{x}} + \overline{{R}_{σ}^{L}}}{2} .$$

### EXAMPLES

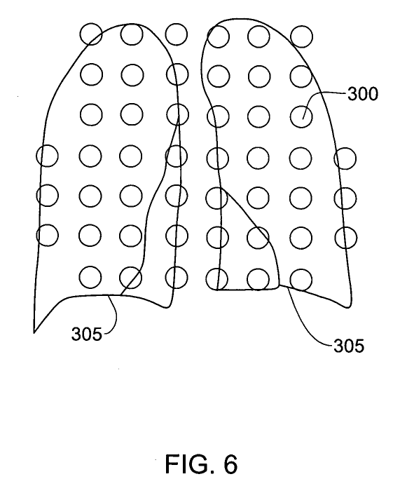

For the regional assessment carried out by the method of the invention, a two-dimensional coordinate system was defined on the subject's back, As shown in FIG. 6, 48 transducers were placed on the individual's back over the lungs at the locations indicated by the circles 300. The curves 305 show the presumed contours of the subject's lungs. The transducers were arranged in a regular orthogonal lattice with a spacing between the transducers in the horizontal and vertical directions of 5 cm. The signals P(xᵢ,t) were then recorded. Each signal was filtered using a 280-350 Hz band-pass filter.

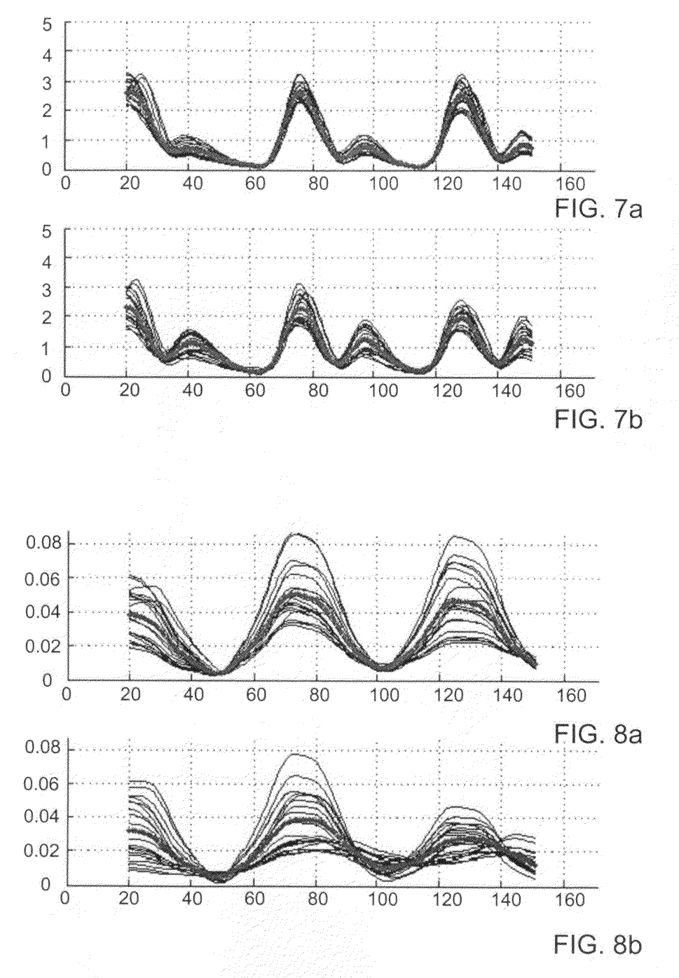

In a first analysis, a regional assessment was performed dividing the set of transducers into two subsets, one subset overlying the left lung, and one subset overlying the left lung. The results are shown in FIGS. 7. to 10.

FIG. 7 shows the functions σ_{fs}^{norm}(xᵢ,k) for the transducers overlying the left lung (FIG. 7a) and the transducers overlying the right lung (FIG. 7b).

FIG. 8 shows the functions R_{σ}(xᵢ,k) for the transducers overlying the left lung (FIG. 8a) and the transducers overlying the right lung (FIG. 8b).

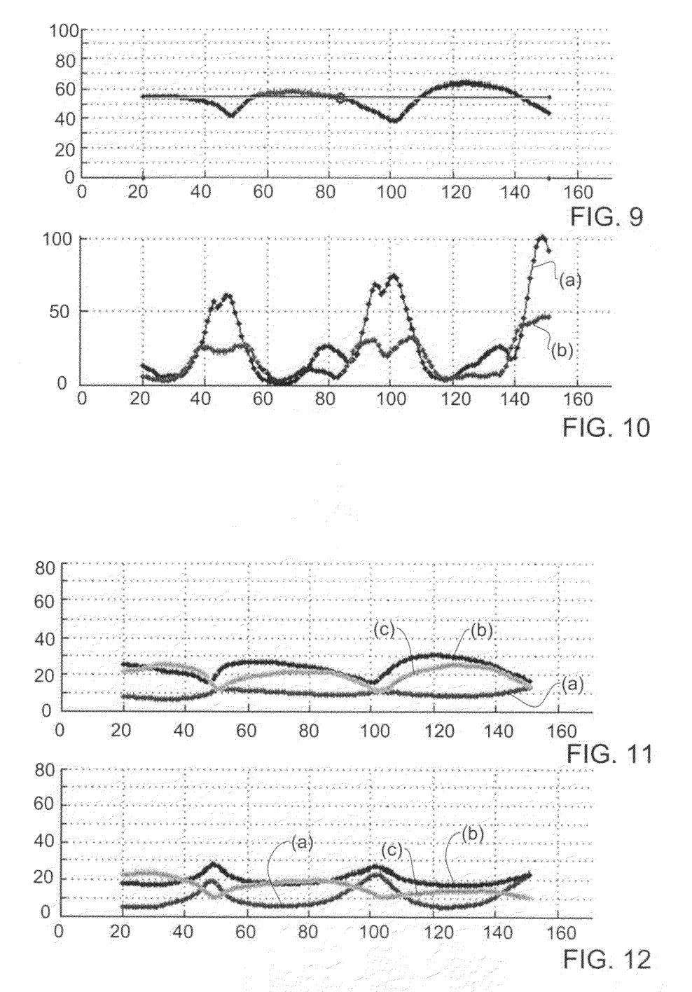

FIG. 9 shows the function R̅_̅{̅σ̅}̅^̅{̅L̅}̅(̅k̅)̅, the function R̅_̅{̅σ̅}̅^̅{̅R̅}̅(̅k̅)̅ being equal to 1− R̅_̅{̅σ̅}̅^̅{̅L̅}̅(̅k̅)̅.

FIG. 10 shows the functions K^{L}(k) (curve (a)) and K^{Ld}(k) (curve (b)).

The overall regional assessment, for the results shown in FIGS. 7 to 10 is 0.55 for the left lung and 0.45 for the right lung.

A regional assessment was also carried out using the signals σ_{fs}^{norm}(xᵢ,k) shown in FIGS. 7a and 7b, in which each lung was divided into three regions (top middle and bottom). FIG. 11 shows the regional assessment of the left lung. Curve (a) shows the regional assessment of the top region, curve (b) shows the regional assessment of the middle region, and curve (c) shows the regional assessment of the bottom region. FIG. 12 shows the regional assessment of the right lung. Curve (a) shows the regional assessment of the top region, curve (b) shows the regional assessment of the middle region, and curve (c) shows the regional assessment of the bottom region.

1. A system for regional assessment in two or more regions of an individual's lungs comprising:
(a) a plurality of N transducers, where n is an integer greater than or equal to 2, each transducer configured to be fixed on a surface of the individual over the thorax, the ith transducer being fixed at a location xᵢ and generating a signal P(xᵢ,t) indicative of pressure waves at the location xᵢ; for i=1 to N; the transducers being divided into subsets, each subset overlying a specific region of the two or more regions; and
(b) a processor configured to:
(i) receive the signals P(xᵢ,t) obtained over a time period and calculate from each signal P(xᵢ,t) an energy assessment signal at the location xᵢ and
(ii) to calculate, for each of the two or more regions, an assessment of each region in a calculation involving the energy assessment signals obtained by transducers overlying the region.

2. The system according to claim 1 wherein the processor is further configured to filter the signals P(xᵢ,t) to produce respective filtered signal S_{f}(xᵢ,t) in order to remove one or more components of the signals which do not arise from respiratory tract sounds.

3. The system according to claim 2 wherein cardiovascular sounds are filtered out.

4. The system according to claim 2 wherein the calculation of the energy assessment signal involves dividing the time period into intervals by a time window and calculating difference signals S_{f}(xᵢ,t)− S̅ₖ(xᵢ), wherein S̅ₖ(xᵢ) is the average value the signal S_{f}(xᵢ,t) in interval k.

5. The system according to claim 4 wherein the calculation of the energy assessment signal involves the algebraic expression |S_{f}(xᵢ,t)− S̅ₖ(xᵢ)|.

6. The system according to claim 5 wherein the calculation of the energy assessment signal involves the expression |S_{f}(xᵢ,t)− S̅ₖ(xᵢ)|^{p} where p is a predetermined constant.

7. The system according to claim 6 wherein p=2.

8. The system according to claim 7 wherein the energy assessment signal is a standard deviation σ(xᵢ,k) of the signal S_{f}(xᵢ,t) in each interval k.

9. The system according to claim 1, wherein the assessment of a region is calculated as a sum of the energy assessment signals of the transducer subset overlying the region.

10. The system according to claim 8 wherein calculation of the energy assessment signal further involves calculation of a normalized standard deviation signal σ_{fs}^{norm}(xᵢ,k)=σ(xᵢ,k)/ σ̅, k=1 . . . nₖ, where nₖ is the number of intervals, and wherein σ̅ is an average value of the standard deviation calculated for all of the intervals,
$$\overline{σ} = \frac{1}{{n}_{k}} {\sum }_{k = 1}^{{n}_{k}} σ ( x i , k ) .$$

11. The system according to claim 10 wherein the calculation of the energy assessment signals further comprises filtering the signals σ^{norm}(xᵢ,k).

12. The system according to claim 11 wherein the filtering is a one-dimensional median filtering to generate filtered normalized standard deviation signals σ_{f}^{norm}(xᵢ,k), for k=1 to nₖ.

13. The system according to claim 12 wherein the calculation of the energy assessment signals further comprises performing extended smoothing of the signals σ_{f}^{norm}(xᵢ,k).

14. The system according to claim 13 wherein the calculation of the energy assessment signals further comprises:
(a) dividing the nₖ-dimensional signals σ_{fs}^{norm}(xᵢ,k) into one or more subintervals by a sliding window having nₛ samples;
(b) calculating the average value of each signal σ_{fs}^{norm}(xᵢ,k) in each subinterval σ̅_{fs}^{norm}(xᵢ,k,s) is calculated, where σ̅_{fs}^{norm}(xᵢ,k,s) is an average value of the signal σ_{fs}^{norm}(xᵢ,k) in a subinterval s of the interval k; and
(c) calculating the energy assessment signal for each subinterval s in the interval k of each signal σ_{fs}^{norm}(xᵢ,k).

15. The system according to claim 14 wherein the calculation of the energy assessment signals further comprises calculating the energy assessment signals R_{σ}(xᵢ,k,s) as the variance of σ_{fs}^{norm}(xₛ,kₛ).

16. A method for regional assessment in two or more regions of an individual's lungs comprising:
(a) receiving a plurality of N signals P(xᵢ,t), where N is an integer greater than or equal to 2, each signal being generated by a transducer fixed on a surface of the individual over the thorax, the ith transducer being fixed at a location xᵢ and generating a signal P(xᵢ,t) indicative of pressure waves at the location xᵢ; for i=1 to N; the transducers being divided into subsets, each subset overlying a specific region of the two or more regions, and the signals P(xᵢ,t) being obtained over a time period;
(b) calculating from each signal P(xᵢ,t) an energy assessment signal at the location xᵢ; and
(c) calculating, for each of the two or more regions, an assessment of each region in a calculation involving the energy assessment signals obtained by transducers overlying the region.
(i)

17. The method according to claim 16 further comprising filtering the signals P(xᵢ,t) to produce respective filtered signals S_{f}(xᵢ,t) in order to remove one or more components of the signals which do not arise from respiratory tract sounds.

18. The method according to claim 17 wherein cardiovascular sounds are filtered out.

19. The method according to claim 17 wherein the calculation of the energy assessment signal involves dividing the time period into intervals by a time window and calculating difference signals S_{f}(xᵢ,t)− S̅ₖ(xᵢ), wherein S̅ₖ(xᵢ) is the average value the signal S_{f}(xᵢ,t) in interval k.

20. The method according to claim 19 wherein the calculation of the energy assessment signal involves the algebraic expression |S_{f}(xᵢ,t)− S̅ₖ(xᵢ)|.

21. The method according to claim 20 wherein the calculation of the energy assessment signal involves the expression |S_{f}(xᵢ,t)− S̅ₖ(xᵢ)|^{p} where p is a predetermined constant.

22. The method according to claim 21 wherein p=2.

23. The method according to claim 22 wherein the energy assessment signal is a standard deviation σ(xᵢ,k) of the signal S_{f}(xᵢ,t) in each interval k.

24. The method according to claim 16, wherein the assessment of a region is calculated as a sum of the energy assessment signals of the transducer subset overlying the region.

25. The method according to claim 23 wherein calculation of the energy assessment signal further involves calculation a normalized standard deviation signal norm σ^{norm}(xᵢ,k)=σ(xᵢ,k)/ σ̅, k=1 . . . nₖ, where nₖ is the number of intervals, and wherein σ̅ is an average value of the standard deviation calculated for all of the intervals,
$$\overline{σ} = \frac{1}{{n}_{k}} {\sum }_{k = 1}^{{n}_{k}} σ ( x i , k ) .$$

26. The method according to claim 25 wherein the calculation of the energy assessment signals further comprises filtering the signals σ^{norm}(xᵢ,k).

27. The method according to claim 26 wherein the filtering is a one-dimensional median filtering, to generate filtered normalized standard deviation signals σ_{f}^{norm}(xᵢ,k), for k=1 to nₖ.

28. The method according to claim 27 wherein the calculation of the energy assessment signals further comprises performing extended smoothing of the signals σ_{f}^{norm}(xᵢ,k).

29. The method according to claim 28 wherein the calculation of the energy assessment signals further comprises:
(a) dividing the nₖ-dimensional signals σ_{fs}^{norm}(xᵢ,k) into one or more subintervals by a sliding window having nₛ samples;
(b) calculating the average value of each signal σ_{fs}^{norm}(xᵢ,k) in each subinterval σ̅_{fs}^{norm}(xᵢ,k,s) is calculated, where σ̅_{fs}^{norm}(xᵢ,k,s) is an average value of the signal σ_{fs}^{norm}(xᵢ,k) in a subinterval s of the interval k; and
(c) calculating the energy assessment signal for each subinterval s in the interval k of each signal σ_{fs}^{norm}(xᵢ,k).

30. The method according to claim 29 wherein the calculation of the energy assessment signals further comprises calculating the energy assessment signals R_{σ}(xᵢ,k,s) as the variance of σ_{fs}^{norm}(xᵢ,kₛ).
